(19) United States
(12) Patent Application Publication
(10) Pub. No.: US 2026/0335528 A1
(43) Pub. Date: Oct. 29, 2026

(54) SURGICAL SUCTION AND IRRIGATION DEVICE HAVING A TUBULAR SHAFT WITH A COUPLING STRUCTURE FOR A ROBOTIC SURGICAL INSTRUMENT AND A FLOW CONTROL STRUCTURE

(71) Applicant: Soonchunhyang University Industry Academy Cooperation Foundation, Asan-si (KR)

(72) Inventor: Daegwang YOO, Yongsan-gu, Seoul (KR)

(21) Appl. No.: 19/641,819

(22) Filed: Apr. 8, 2026

(30) Foreign Application Priority Data
Apr. 23, 2025 (KR) 10-2025-0052755
Mar. 17, 2026 (KR) 10-2026-0048175

(51) Int. Cl. A61M 1/00; A61B 34/30; A61M 25/01

(52) U.S. Cl. CPC A61M 1/87; A61B 34/30; A61M 1/774; A61M 25/0133; A61B 2034/301; A61B 2217/005; A61B 2217/007

(57) ABSTRACT

An example surgical suction and irrigation device includes a tubular shaft with a robotic surgical instrument coupling structure and a flow rate control structure. The device includes a main body, an extension portion fluidically connected to the main body and at least partially elastically bendable so as to extend to a surgical site, and an instrument coupling portion provided on an outer side of a distal portion of the extension portion and configured to be selectively coupled with a jaw or grip member of a robotic surgical instrument or a laparoscopic instrument, in which the main body is configured to perform suction or irrigation through the extension portion, and a position of the distal portion of the extension portion may be controlled according to position control of the jaw or the grip member when the jaw or the grip member is coupled to the instrument coupling portion.

### CROSS-REFERENCE TO RELATED APPLICATIONS

This application claims the benefit of and priority to Korean Patent Application No. 10-2025-0052755 filed on Apr. 23, 2025, and Korean Patent Application No. 10-2026-0048175 filed on Mar. 03, 2026, in the Korean Intellectual Property Office, the disclosures of which are incorporated herein by reference in their entireties.

### BACKGROUND

### Field

The present disclosure relates to a surgical suction and irrigation device usable in minimally invasive surgery such as robot surgery, laparoscopic surgery, and thoracoscopic surgery. More particularly, the present disclosure relates to a surgical suction and irrigation device configured to suction body fluids and supply an irrigation fluid through a tubular shaft extending to a surgical site, in which a distal portion of the tubular shaft is coupled to a jaw or a grip member of a robotic surgical instrument to be position-controlled, and a flow rate is controllable as needed.

### Description of the Related Art

In minimally invasive surgery such as robot surgery, laparoscopic surgery, and thoracoscopic surgery, bleeding, body fluids, smoke, and tissue debris continuously occur within a narrow working space, and thus maintaining a clear field of view at a surgical site is very important. Even a slight deterioration in visibility may delay identification of a bleeding point or an anatomical boundary, which directly affects accuracy and speed of surgery.

For these reasons, a suction and irrigation device has been widely used in surgical settings to aspirate blood or body fluids and, when necessary, to discharge saline or the like to wash a surgical site. A conventional surgical suction and irrigation device generally includes a main body, a flow path connected to an external suction source and an irrigation fluid supply source, and a distal-end portion inserted into a surgical site, and has a structure in which a user selectively performs suction or irrigation by manually operating a button or a lever.

However, since the conventional surgical suction and irrigation device generally has a rigid shaft, there is a limitation in precisely accessing or flexibly adjusting a position along a curved path in accordance with movement of a robot instrument in a narrow and complex space such as robot surgery. In particular, in robot surgery, an approach direction to a surgical site is diverse and articulation motion of a robot instrument is actively utilized, and thus it is difficult to flexibly cope with such an environment using only a rigid shaft structure having high rigidity.

In order to compensate for such limitations, a flexible suction instrument has been considered in some cases. However, conventional flexible instruments are not configured to be coupled to a jaw of a robotic surgical instrument and controlled together, but are often separately held or positioned by an assistant. As a result, one hand of a surgeon or an additional operation by an assistant is required, thereby reducing a degree of freedom in operating other instruments during surgery and causing inconvenience in performing delicate surgical procedures. In other words, it is not sufficient that a shaft is merely flexible, and there is a need for a structure in which the flexible distal end is cooperatively position-controlled together with a robotic surgical instrument.

In addition, the conventional device exhibits various limitations in terms of usability in an actual surgical process. A representative problem is that an irrigation function is generally of a momentary operation type. That is, since saline or an irrigation fluid is supplied only while the user presses a button, the user should continuously press the button with a finger to maintain irrigation for a certain period of time. This may not be a significant problem for a short duration, but causes considerable inconvenience in a situation where a temperature of a cut surface needs to be lowered or a tissue surface needs to be maintained in a wet state continuously.

For example, in a surgery such as hepatectomy in which heat generation is large during a cutting process and a cut surface is easily dried or carbonized, continuous irrigation is often clinically advantageous. However, in the conventional structure, one finger is continuously constrained to button operation, resulting in reduced freedom of other operations, increased finger fatigue during long-term repeated use, and variation in flow rate depending on a pressing force of the button. As a result, the more continuous irrigation is required, the greater an operational burden imposed on the user due to such structural limitations.

Further, in the conventional device, flow rate control is often not sufficiently precise. During surgery, there are cases where vigorous washing is required, but there are also many cases where only a necessary region needs to be gently moistening without excess fluid. Nevertheless, many conventional devices merely open and close a flow path by pressing a button, making it difficult to stably maintain a desired flow rate. As a result, a user must rely on intuition by pressing the button strongly or weakly, making it difficult to reproduce a consistent irrigation condition. This issue makes it difficult to consistently manage a state of a surgical site and also indicates a limitation in that results vary depending on a level of skill of a user. In other words, although the conventional technology provides an irrigation function itself, it is insufficient in terms of continuity, stability, and fine controllability.

There are also inconveniences in a distal-end portion structure. In actual surgery, an appropriate distal end shape may vary depending on an approach path, a position of a target site, an arrangement of surrounding tissues, a narrow working space, and required directionality and flexibility. In some cases, a flexible distal end capable of following a curved path according to articulation motion of a robotic surgical instrument is advantageous, whereas in other cases, a rigid distal end having good directionality and support is more suitable. However, in the conventional suction and irrigation device, a distal end shape is fixed or an entire device needs to be replaced in order to change a distal end, making it difficult to flexibly respond during surgery. Such a method of replacing the entire device increases the number of instruments to be prepared, requires additional replacement time, and may interrupt a flow of surgery.

Accordingly, although the conventional surgical suction and irrigation devices are capable of performing basic suction and irrigation functions, they have limitations in simultaneously satisfying requirements frequently encountered in actual surgical settings, including improved accessibility by a flexible shaft, position control through coupling with a jaw of a robotic surgical instrument, maintenance of continuous irrigation, stable flow rate control, reduction of manual operation burden, selection of a distal end suitable for a surgical situation, and rapid yet stable replacement of the distal end. That is, a core problem of the conventional technology is not merely whether a fluid can be aspirated and sprayed, but how conveniently, accurately, and appropriately such functions can be used by a surgeon in a narrow and complex minimally invasive surgical environment, particularly in a robot surgical environment.

### SUMMARY

An object of the present disclosure is to provide a surgical suction and irrigation device having a tubular shaft that is position-controlled in cooperation with a robotic surgical instrument in a robot surgery environment. Another object of the present disclosure is to provide a surgical suction and irrigation device capable of more efficiently performing suction and irrigation with respect to a surgical site, controlling a flow rate as needed, and selectively applying a distal end configuration according to a surgical situation.

In order to achieve the above-described objects, according to the present disclosure, there is provided a surgical suction and irrigation device having a tubular shaft with a robotic surgical instrument coupling structure and a flow rate control structure. The device includes: a main body; an extension portion fluidically connected to the main body and at least partially elastically bendable so as to extend to a surgical site; and an instrument coupling portion provided on an outer side of a distal portion of the extension portion and configured to be selectively coupled with a jaw or grip member of a robotic surgical instrument, a laparoscopic instrument, or a thoracoscopic instrument, in which the main body is configured to perform suction or irrigation through the extension portion, and a position of the distal portion of the extension portion is controlled according to position control of the jaw or the grip member when the jaw or the grip member is coupled to the instrument coupling portion.

According to an embodiment of the present disclosure, the main body may include a first input button and a second input button corresponding to a suction function and an irrigation function, respectively.

According to an embodiment of the present disclosure, the main body may further include a first rotating lever and a second rotating lever configured to transmit actuating forces of the first input button and the second input button to a fluid control portion of the main body, and the first and second rotating levers may each include a rotating support and an inclined acting surface to convert a button pressing displacement into a fluid control actuation displacement.

According to an embodiment of the present disclosure, the instrument coupling portion may include a distal connecting member provided at the distal portion of the extension portion, and a fitting-type coupling portion formed in the distal connecting member configured to allow insertion and removal of an end portion of the jaw or grip member.

According to an embodiment of the present disclosure, the fitting-type coupling portion may be formed in a pocket shape.

According to an embodiment of the present disclosure, the fitting-type coupling portion may include a toothed portion on the inner side, outer side, or both sides to increase a frictional force with the jaw or grip member.

According to an embodiment of the present disclosure, the toothed portion may have a triangular or right-triangular cross-section to prevent detachment after insertion.

According to an embodiment of the present disclosure, the instrument coupling portion may further include a through-type coupling portion configured to allow the end portion of the jaw or grip member to pass through and be separated.

According to an embodiment of the present disclosure, the through-type coupling portion may include a latch projection line to prevent the jaw or grip member from easily disengaging after passing through.

According to an embodiment of the present disclosure, a distal end of the extension portion may include at least one through hole.

According to an embodiment of the present disclosure, the extension portion may be configured to extend to the surgical site through a port or hole separate from a primary port through which a robotic arm passes during robotic surgery.

According to an embodiment of the present disclosure, the extension portion may be formed as a replaceable distal-end portion detachably coupled to the main body.

According to an embodiment of the present disclosure, the main body may include a distal-end coupling portion to which the replaceable distal-end portion is coupled, the distal-end coupling portion may include a main-body-side female coupling portion, a rotary locking sleeve, and a distal-end insertion guide portion, and the replaceable distal-end portion may be inserted axially into the main-body-side female coupling portion, and then, be configured such that a coupled state is maintained by rotation of the rotary locking sleeve.

According to an embodiment of the present disclosure, the replaceable distal-end portion may include a proximal connector portion inserted into the main-body-side female coupling portion, and a sealing portion may be interposed between the proximal connector portion and the main-body-side female coupling portion for fluid sealing.

According to an embodiment of the present disclosure, the replaceable distal-end portion may include a plurality of distal-end portions having a common coupling interface, and the plurality of distal-end portions may include a flexible tube-type distal-end portion with an instrument coupling portion and a rigid tubular distal-end portion.

According to an embodiment of the present disclosure, the main body may further include a dial-type continuous irrigation control portion configured to mechanically maintain an actuation input state corresponding to the irrigation function and vary an irrigation flow rate according to a rotation amount of a dial.

According to an embodiment of the present disclosure, the dial-type continuous irrigation control portion may include a control dial rotatably arranged on a side surface of the main body and a rotational transmission portion configured to transmit rotation of the control dial to an internal actuation system.

According to an embodiment of the present disclosure, the rotational transmission portion may be configured to limit return of the actuation input state corresponding to the irrigation function so that a continuous irrigation state is maintained even when a user releases an input portion corresponding to the irrigation function, and a support position or pressing force of the actuation system corresponding to the irrigation function may be changed according to the rotation amount of the control dial so that an opening amount of an irrigation flow path is variable.

According to an embodiment of the present disclosure, the main body may further include a fluid control and delivery portion including a main valve cartridge, a plurality of external fluid connection ports, and a front flow path delivery assembly extending from the main valve cartridge toward the extension portion.

The main valve cartridge may include a suction control portion and an irrigation control portion in a single cartridge body, and the front flow path delivery assembly may be configured to form a suction flow path and an irrigation flow path that are separated from each other, and to be elastically supported or to have a connection state thereof stabilized, by an elastic support and retaining member.

According to the present disclosure, in the surgical suction and irrigation device, the suction function and the irrigation function can be performed more stably and intuitively according to an intention of a surgeon, thereby allowing a surgical field of view obscured by bleeding, body fluids, smoke, and tissue debris to be more rapidly secured. In particular, by adopting a flexibly bendable distal-end structure, accessibility to a surgical site and a degree of freedom in position adjustment in a narrow and complex space can be improved, and the device can be more effectively used in a robot surgery environment.

Further, in the present disclosure, since the distal end is coupled to the jaw of the robotic surgical instrument or the laparoscopic instrument and is position-controlled together, a burden on a surgeon to separately hold and manipulate an additional flexible suction instrument can be reduced. Accordingly, the suction and irrigation functions can be performed more naturally even during robot surgery, and while securing a field of view of a bleeding site, treatment using other surgical instruments can be more efficiently performed in parallel. In particular, there is an advantage in that operability and precision, which are important in robot surgery, can be improved.

In addition, since the irrigation function is not limited to a momentary operation but can be continuously maintained for a required period of time, it becomes easier to maintain a surgical site or a cut surface in a consistently irrigated and cooled state, thereby improving continuity of observation and treatment of the surgical site. Further, since a flow rate can be controlled in a state where the irrigation function is continuously maintained, an amount of an irrigation fluid supplied can be more finely adjusted according to a condition of a surgical site or a surgical stage.

Accordingly, while reducing the view obstruction caused by excessive spraying of the irrigation fluid, a required irrigation effect can be stably secured, and a burden on a user of continuously pressing a button can be reduced, thereby alleviating finger fatigue and inconvenience of repetitive operation. Therefore, more stable usability can be provided even in a situation requiring a long operation time or delicate surgical manipulation.

Further, since different types of distal-end portions can be selectively coupled to and used with a single main body, an appropriate distal end can be selected according to a position of a surgical site, an approach path, narrowness of a working space, required directionality, and rigidity or flexibility. For example, when flexibly approaching using articulation motion of the robotic surgical instrument, a flexible distal-end portion may be advantageous, and when greater directionality or a rigid shaft is required, a rigid distal-end portion may be advantageous. Accordingly, various surgical situations that are difficult to address with a single distal end shape can be more flexibly handled, and inconvenience of replacing an entire device in order to change a distal end shape can be reduced.

Furthermore, the present disclosure can be mainly applied to robot surgery and can also be applied, as necessary, to other minimally invasive surgeries such as laparoscopic surgery or thoracoscopic surgery. Accordingly, securing a field of view, cleaning of a surgical site, identification of a bleeding site, and assistance in treatment can be more smoothly performed, and overall surgical convenience and work efficiency can be improved. Therefore, the present disclosure has an effect of overall improving operability, continuous usability, adaptability to various situations, and practical reliability of the surgical suction and irrigation device.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a schematic view illustrating a state in which a surgical suction and irrigation device according to one embodiment of the present disclosure is coupled to a jaw of a robotic surgical instrument and is disposed at a surgical site through a port.

FIG. 2 is a side view illustrating a state before the jaw is coupled to a fitting-type coupling portion of a robot instrument coupling portion in the surgical suction and irrigation device according to one embodiment of the present disclosure.

FIG. 3 is a side view illustrating a state in which the jaw is inserted into and coupled to a fitting-type coupling portion of the robot instrument coupling portion in the surgical suction and irrigation device according to one embodiment of the present disclosure.

FIG. 4 is a cross-sectional view illustrating an engagement relationship among the fitting-type coupling portion, a toothed portion, and the jaw in the surgical suction and irrigation device according to one embodiment of the present disclosure.

FIG. 5 is a side view illustrating a state in which a jaw is coupled to a through-type coupling portion in a surgical suction and irrigation device according to another embodiment of the present disclosure.

FIG. 6 is an exploded perspective view illustrating an overall configuration of the surgical suction and irrigation device according to one embodiment of the present disclosure.

FIG. 7 is a partially enlarged perspective view illustrating an arrangement of an upper button and a side control dial in the surgical suction and irrigation device according to one embodiment of the present disclosure.

FIG. 8 is an exploded perspective view illustrating an arrangement relationship between internal components and a distal-end coupling portion with respect to a first housing in the surgical suction and irrigation device according to one embodiment of the present disclosure.

FIG. 9 is an exploded perspective view illustrating internal components of the surgical suction and irrigation device according to one embodiment of the present disclosure from another direction.

FIG. 10 is an exploded perspective view illustrating an assembly relationship among a replaceable distal end, the distal-end coupling portion, and an internal configuration of a main body in the surgical suction and irrigation device according to one embodiment of the present disclosure.

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

Hereinafter, the detailed description for implementing the present disclosure will be described with reference to the accompanying drawings. Moreover, in explaining the present disclosure, detailed descriptions of known functions, which are self-evident to those skilled in the art and could unnecessarily obscure the gist of the present disclosure, are omitted.

A surgical suction and irrigation device according to the present disclosure includes a robotic instrument coupling portion 10, a main body portion 100, an operation input portion 200, a fluid control and delivery portion 300, a dial-type continuous irrigation control portion 400, a distal-end coupling portion 500, and a replaceable distal-end portion 600.

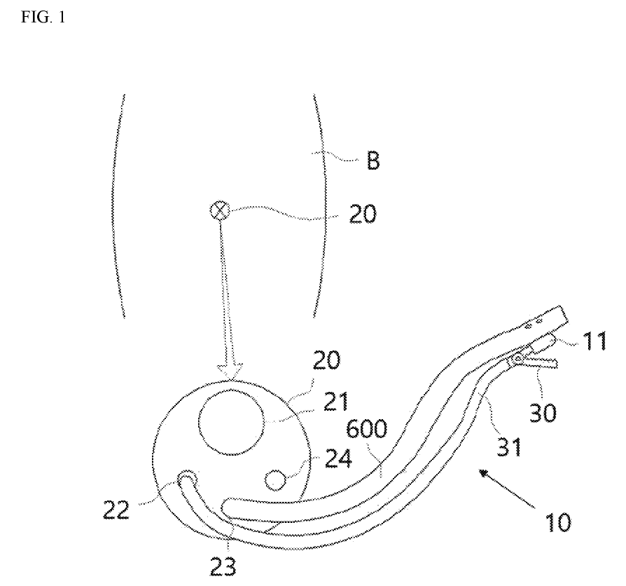

The robotic instrument coupling portion 10 is illustrated in FIGS. 1-5, and the operation input portion 200 to the replaceable distal-end portion 600 are illustrated in FIGS. 6-10.

FIG. 1 schematically illustrates the robotic instrument coupling portion 10 in a state of use in an actual surgical environment, along with a glove port 20 installed in a body B, a central primary port 21 of the glove port 20, peripheral first, second, and third auxiliary ports 22, 23, and 24, the replaceable distal-end portion 600, and a grip member and jaw 30 of a robot instrument selectively coupled to the robotic instrument coupling portion 10. The jaw 30 may be connected to a distal end of a jaw connection line 31. The jaw connection line 31 may be operated so that the position of the jaw 30 is changed by the control of the surgical robot.

Here, the glove port 20 is a port structure installed in an incision formed in the body B. The primary port 21 may function as a central primary port, and the first to third auxiliary ports 22, 23, and 24 may function as peripheral ports through which auxiliary instruments or the replaceable distal-end portion 600 may selectively pass. In particular, in FIG. 1, it can be understood that the replaceable distal-end portion 600 is extended into the body through the second auxiliary port 23.

The robotic instrument coupling portion 10 is provided at a distal end of the replaceable distal-end portion 600, particularly a flexibly bendable tube-type distal-end portion, and is configured to be selectively coupled with a jaw 30 or a grip member of the robotic surgical instrument or laparoscopic instrument. Conventional suction and irrigation devices generally have rigid shafts, which limit access to narrow and curved spaces and precise directional control in a robotic surgery environment. However, the present disclosure includes the distal-end portion that is flexibly bendable and the robotic instrument coupling portion 10, thereby allowing a distal end of the distal-end portion to be more easily guided to a desired surgical site using position control of the robotic instrument. In other words, a surgeon may perform suction and irrigation while controlling the distal end position through the jaw 30 or grip member of the robot instrument, without directly holding a separate suction instrument by hand.

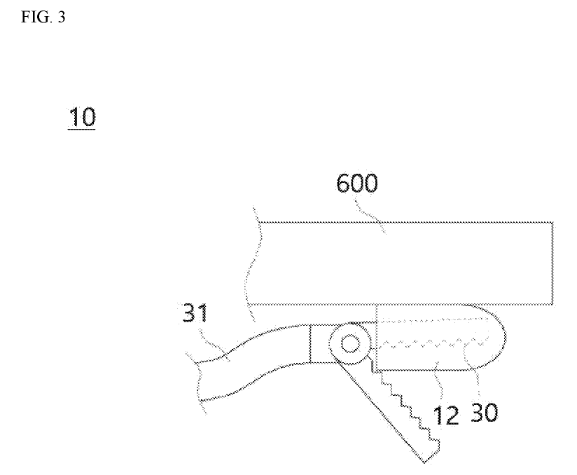

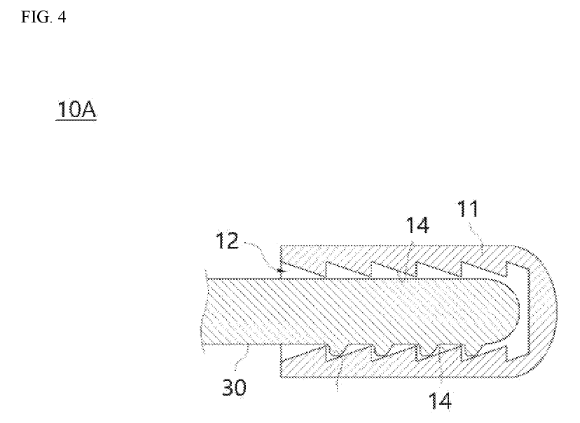

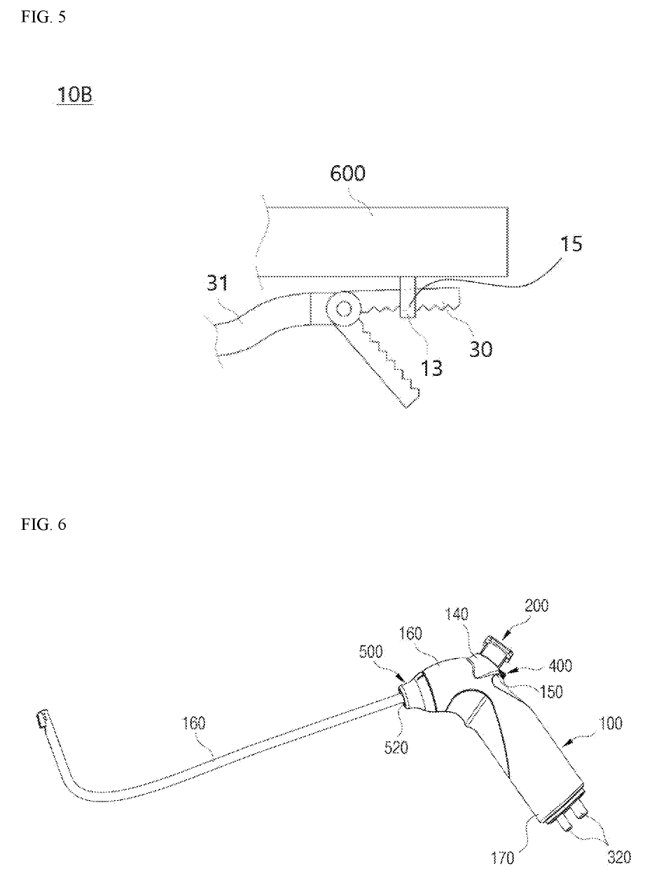

The robotic instrument coupling portion 10 may include a distal connecting member 11. The distal connecting member 11 is provided at a distal end of the replaceable distal-end portion 600 (FIGS. 1 and 6) and forms a base structure on which a fitting-type coupling portion 12 (FIGS. 3 and 4) or a through-type coupling portion 13 (FIG. 5) is formed or supported. In other words, the distal connecting member 11 can be understood as a support body that stably maintains the coupling structure that actually meshes with the robot instrument at the distal end. The distal connecting member 11 may be disposed on an outer side of, or at a position adjacent to, a distal passage through which a fluid actually flows, and may be configured to perform a mechanical coupling function with the robotic instrument without substantially interfering with suction and discharge functions of the fluid.

As illustrated in FIG. 1, the distal connecting member 11 may be formed at a position where the jaw 30 of the robot instrument directly approaches the most distal end of the replaceable distal-end portion 600, thus functioning as a mediating structure that allows the posture change of the robot instrument to directly translate into position changes of the distal end.

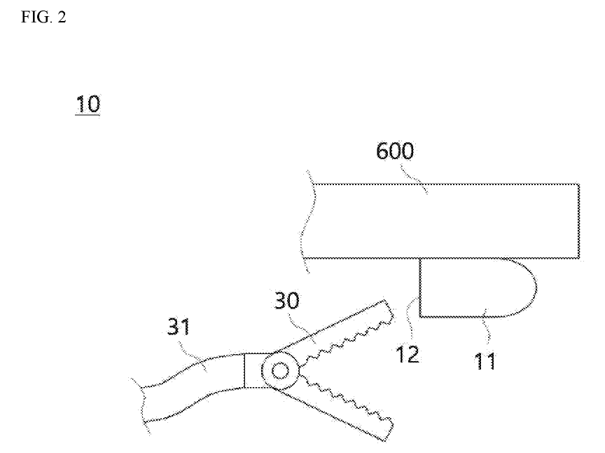

Referring to FIG. 4, the robotic instrument coupling portion 10 may include a fitting-type coupling portion 12 as a first embodiment (10A). The fitting-type coupling portion 12 is a coupling space formed to allow at least a part of the jaw 30 or the grip member of the robot instrument to be inserted and separated, which can be confirmed in FIGS. 2, 3, and the embodiment of FIG. 4 (10A). The fitting-type coupling portion 12 may, for example, be formed in a pouch shape or pocket shape, with one end open and the other closed, or, if necessary, both ends open.

During use, at least a portion of the jaw 30 of the robot instrument is inserted into the fitting-type coupling portion 12, and while maintaining this insertion state, the position of the distal end may be controlled according to the movement of the robot instrument together with the distal connecting member 11. Therefore, the fitting-type coupling portion 12 may serve as a “slot for insertion” of the jaw 30 of the robot instrument and a temporary coupling means for securing the distal end to the robot instrument.

A toothed portion 14 may be formed in the fitting-type coupling portion 12. The toothed portion 14 is configured to increase a frictional force and an engagement force with the jaw 30 or grip member of the robot instrument and may be formed on the inner side, outer side, or both sides of the fitting-type coupling portion 12. In the cross-sectional example of FIG. 4, the toothed portion 14 is formed along the surface contacting the inserted jaw 30 to prevent the jaw 30 from easily disengaging after coupling.

The toothed portion 14 may have a cross-sectional shape of a triangle, right triangle, or wedge, such that after the jaw 30 of the robot instrument is inserted into the fitting-type coupling portion 12, insertion in the forward direction is relatively easy, while disengagement in the opposite direction is restricted. In other words, the toothed portion 14 is understood as a frictional and engagement structure that enhances coupling stability rather than a simple surface irregularity.

Referring to FIG. 5, the robotic instrument coupling portion 10 may include a through-type coupling portion 13 as another embodiment (10B). The through-type coupling portion 13 is formed to allow a part of the jaw 30 or grip member of the robot instrument to pass through the coupling structure, corresponding to the embodiment (10B) in FIG. 5. That is, while the fitting-type coupling portion 12 is configured to receive the jaw 30 therein, the through-type coupling portion 13 is configured to allow the jaw or a part thereof to pass through the coupling portion so that position control is enabled. The through-type coupling portion 13 may be formed in a plate shape or a bracket shape, and may be supported by the distal connecting member 11 such that a distal end moves together even after the jaw 30 of the robotic instrument passes therethrough.

A latch projection line 15 may be provided in the through-type coupling portion 13. The latch projection line 15 is a locking structure that prevents the penetrated jaw 30 from easily disengaging and may be formed along one side or the inner edge of the through-type coupling portion 13. As illustrated in FIG. 5, after the jaw of the robot instrument passes through the through-type coupling portion 13, the jaw is disposed to correspond to the latch projection line 15, reducing the risk of the distal end simply sliding off. Therefore, the latch projection line 15 may function as an auxiliary fixing structure to maintain a more stable coupling state in the through-type coupling method. In other words, when the through-type coupling portion 13 is a “passing structure,” the latch projection line 15 is understood as a “disengagement prevention structure” after passing.

The main body portion 100 includes a first housing 110, a second housing 120, an internal accommodation space 130, an upper button mounting portion 140, a side dial exposure and support portion 150, a front coupling support portion 160, and a rear fluid port support portion 170.

The first housing 110 is a half body forming one outer side of the main body portion 100, and is configured to define an outer shape of a handle grasped by the surgeon. The first housing 110 is not merely an exterior cover but provides a reference surface for positioning internal components while maintaining the shape of the main body portion 100. At least a part of the upper button mounting portion 140, the side dial exposure and support portion 150, the front coupling support portion 160, and the rear fluid port support portion 170 may be formed or supported in the first housing 110.

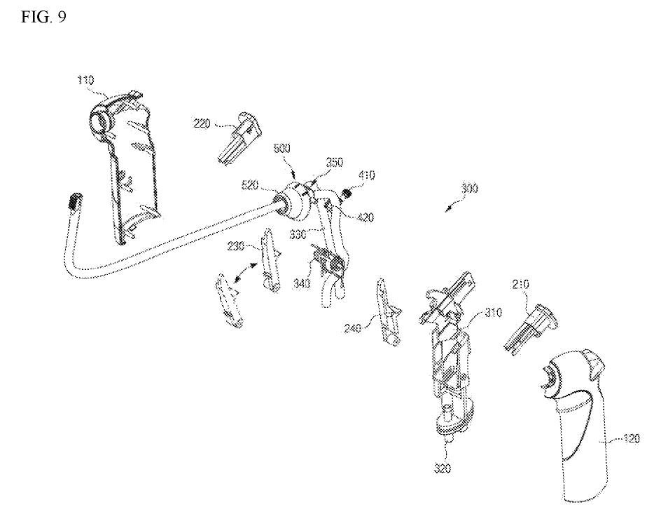

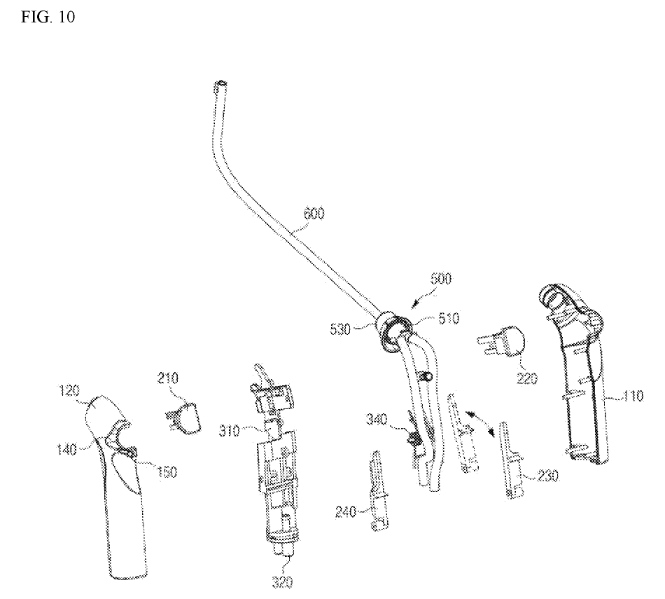

The second housing 120 is the other half body corresponding to the first housing 110, and is configured to be coupled with the first housing 110 to define an overall outer shape of the main body portion 100. The first housing 110 and the second housing 120 form a pair of shells facing each other, and when coupled, define a closed space therein capable of accommodating various actuation components and fluid control components. As illustrated in FIGS. 9 and 10, the second housing 120 is disposed to cover the main body from an opposite side of the first housing 110, and when assembled, engages with the first housing 110 to secure rigidity of the handle and stably accommodate internal components.

The internal accommodation space 130 is formed inside the main body portion 100 when the first housing 110 and the second housing 120 are combined, in which at least a part of the operation input portion 200, the fluid control and delivery portion 300, and the dial-type continuous irrigation control portion 400 are arranged. The internal accommodation space 130 is not merely an empty space inside a handle, but a functional space in which a structure for transmitting a force when a button is pressed, a structure for controlling suction and irrigation, and a structure for transmitting rotation of a dial to an internal actuation system are accommodated.

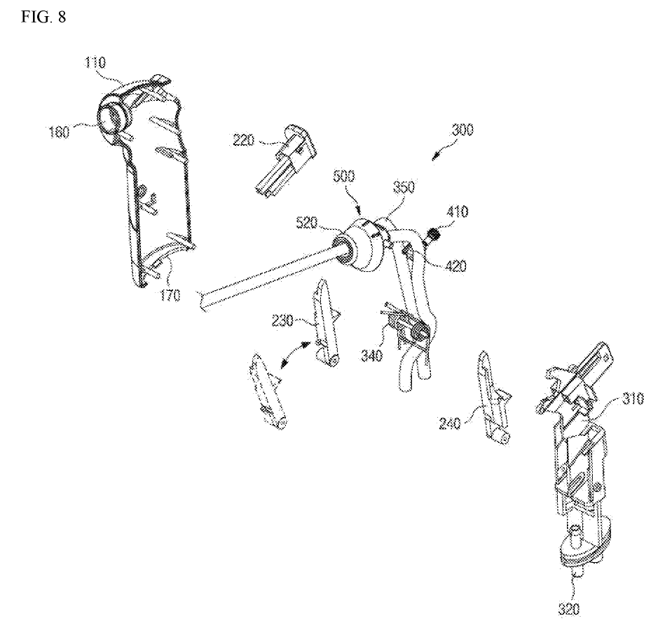

As seen from the inside of the first housing 110 in FIG. 8, the internal accommodation space 130 is not merely an empty space but an internal area designed to allow each component to be placed in its proper position. Since the internal accommodation space 130 may accommodate the main valve cartridge 310, the rotating levers 230 and 240, and a rotation transmission portion 420 operatively associated with the control dial 410, the internal accommodation space 130 may be regarded as an accommodation region within the main body that actually contains the operating structure of the device of the present disclosure.

The upper button mounting portion 140 is formed on the upper portion of the main body portion 100 and supports and allows operation of a first input button 210 and a second input button 220.

The upper button mounting portion 140 is not limited to a flat surface on which buttons are merely placed, but may provide a structure that supports each button so as to be stably positioned at a predetermined location without wobbling and guides the button to smoothly move downward when pressed. Accordingly, the upper button mounting portion 140 may be regarded as a base support structure that maintains a consistent operational feel even when the user repeatedly presses the buttons and allows an actuating force of the buttons to be stably transmitted to an internal rotating lever or a fluid control structure.

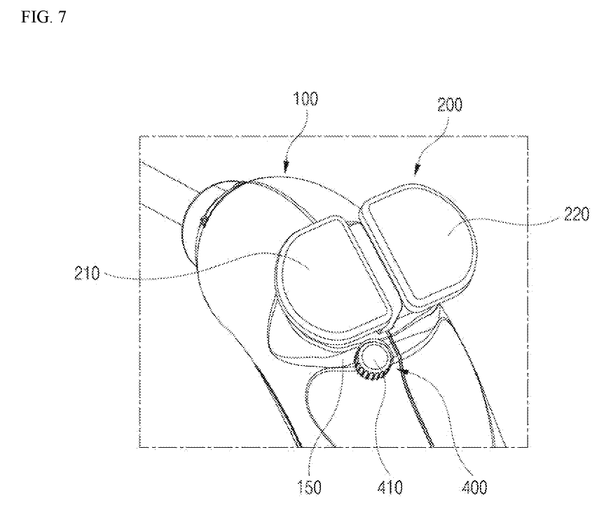

The upper button mounting portion 140 may be formed in connection with the upper shape of the first housing 110 or the second housing 120 and may form a support area surrounding the buttons when both housings are combined. Referring to FIG. 7, the upper button mounting portion 140 corresponds to the area around an upper protrusion where the two buttons are arranged side by side, which is also illustrated in FIG. 10 where the buttons rest on the upper portion of the second housing 120.

The side dial exposure and support portion 150 is formed on a side surface of the main body portion 100 to allow at least a part of the control dial 410 to be exposed to the outside, while simultaneously supporting the control dial 410 to be rotatable.

The side dial exposure and support portion 150 prevents the dial from retracting into the main body or disengaging outward while providing a fixed position so that the rotation of the control dial 410 can be transmitted to the rotation transmission portion 420. Therefore, the side dial exposure and support portion 150 is not merely a hole or an opening, but may be regarded as a support interface that connects an externally operated dial with an internal actuation system.

Based on FIG. 7, the side dial exposure and support portion 150 is located below and near the side surface of the upper button mounting portion 140, forming a region in which the control dial 410 is partially exposed externally. In FIG. 10, the position and supporting configuration where the dial may be disposed on the side surface of the second housing 120 are also illustrated.

As described above, the side dial exposure and support portion 150 not only ensures external operability of the control dial 410, but also supports the control dial 410 such that the rotational axis of the dial does not wobble, and maintains a constant relative position with respect to the internal actuation system, thereby enabling stable implementation of continuous irrigation maintenance and flow rate control functions. Accordingly, the side dial exposure and support portion 150 is not merely a portion that exposes the dial to the outside, but is a main-body-side structure configured to arrange, support, and guide the dial such that the dial can perform its intended function.

The front coupling support portion 160 is provided on the front side of the main body portion 100 to stably dispose and support the distal-end coupling portion 500. More specifically, the front coupling support portion 160 may support the main-body-side female coupling portion 510, the rotary locking sleeve 520, and the distal-end insertion guide portion 530 so as to maintain predetermined positions thereof with respect to the main body portion 100.

Accordingly, the replaceable distal-end portion 600 may be accurately inserted toward the main body portion 100 along the direction guided by the front coupling support portion 160, and after insertion, the coupling state may be stably maintained by the rotation of the rotary locking sleeve 520. In addition, the front coupling support portion 160 provides counterforce against an axial insertion force, withdrawal force, and rotational force generated during detachment and attachment of the distal end, reducing wobbling or misalignment of the distal-end coupling portion 500.

Furthermore, the front coupling support portion 160 stabilizes the relative position between the front flow path delivery assembly 330 and the distal-end coupling portion 500, thereby ensuring a more stable fluid communication and sealing state with the replaceable distal-end portion 600. The front coupling support portion 160 is not merely a portion forming a front opening, but may function as a reference support portion that positions an exact coupling location of the replaceable distal-end portion 600 and ensures mechanical stability of a front coupling structure even during repeated attachment and detachment and use.

The rear fluid port support portion 170 is provided on the rear side of the main body portion 100 to stably support a plurality of external fluid connection ports 320 while exposing them externally. The external fluid connection port 320 may be connected to a suction source and an irrigation fluid supply source, respectively, and the rear fluid port support portion 170 may allow the ports to be arranged while maintaining predetermined spacing and orientation so as to prevent misalignment of a connection with the main valve cartridge 310.

In addition, the rear fluid port support portion 170 allows a pulling force or a twisting force applied to the external fluid connection port 320 during connection or disconnection of an external tube to be received by the main body portion 100, thereby suppressing wobbling or separation of the external fluid connection port 320 and reducing a possibility of leakage at a connection portion. In other words, the rear fluid port support portion 170 is not merely a rear hole through which the port passes, but can be understood as a rear support structure that simultaneously ensures positional alignment, mechanical fixation, assembly stability, and fluid connection reliability of the external fluid connection port 320.

The operation input portion 200 includes the first input button 210, the second input button 220, the first rotating lever 230, and the second rotating lever 240.

The first input button 210 and the second input button 220 are representative external control portions forming the operation input portion 200 and are disposed on the upper button mounting portion 140 of the main body portion 100 so that the surgeon may directly press them with a finger while gripping the main body portion 100.

Referring to FIG. 7, the two buttons are disposed side by side on the upper portion of the main body, and referring to FIGS. 8 to 10, it can be confirmed that each button is seated or inserted into the button mounting position formed on the upper portion of the main body portion 100. Therefore, these buttons are not merely externally protruding decorative elements but are input portions that transmit the user’s operating force to the internal actuation system.

The first input button 210 is disposed on one side of the upper button mounting portion 140 and may be configured to transmit an operating force to the first rotating lever 230 or a corresponding internal actuation system when being pressed. When the user presses the first input button 210 downward, the pressing force is transmitted internally, causing the corresponding control portion of the fluid control and delivery portion 300 to operate.

In this process, the first input button 210 is guided and supported by the upper button mounting portion 140, so that even when repeatedly pressed by the user, the button does not excessively wobble or tilt and may perform relatively stable vertical actuation. In other words, the first input button 210 can be understood as both the starting point of external finger operation and an input transmission member that actuates the internal rotating lever and valve system.

The second input button 220 is another button disposed adjacent to the first input button 210 and may be configured to transmit an operating force to the second rotating lever 240 or a corresponding internal actuation system. Therefore, when the user presses the second input button 220, a different fluid function from that of the first input button 210 may be selectively operated.

The first input button 210 and the second input button 220 may be configured to correspond to different fluid functions, for example, a suction function and an irrigation function, respectively. That is, one button induces operation for suction, and the other button induces operation for irrigation, thereby allowing the surgeon to intuitively select and perform the required function during surgery.

In addition, the first input button 210 and the second input button 220 are arranged side by side so that they can be pressed independently, while each is linked to the corresponding internal actuation system to perform different control operations. In particular, in the present disclosure, the input button corresponding to the irrigation function may be interlocked with the control dial 410 and the rotation transmission portion 420. Therefore, the button serves not only as a momentary press input but also as a start input for forming a continuous irrigation state.

That is, when the control dial 410 is rotated in a state where the button corresponding to the irrigation function is pressed, the internal actuation system is mechanically maintained so that the irrigation state may continue even if the user releases the button. In this case, the button retains its significance as a basic input member that receives the initial user input and transmits it to the internal actuation system.

The first rotating lever 230 is rotatably disposed in the internal accommodation space 130 of the main body portion 100, adjacent to the main valve cartridge 310, serving as a force transmission member that conveys the pressing operation of the first input button 210 to the fluid control and delivery portion 300.

More specifically, the first rotating lever 230 is arranged to rotate about a rotating support on one side, contacting the first input button (210) at its upper or one side, and contacting the corresponding actuation portion of the main valve cartridge 310 at its opposite or intermediate portion. Accordingly, when the user presses the first input button 210, the pressing force is transmitted to the first rotating lever 230, which rotates about the rotating support and, through its inclined acting surface or acting portion, pushes or applies pressure to the corresponding fluid control portion of the main valve cartridge 310.

As a result, the first rotating lever 230 may function as a mechanical conversion portion that transforms the relatively short linear motion of the button into a rotational and pressing motion suitable for actuating the main valve cartridge 310. Furthermore, by using the first rotating lever 230, the actuating force may be reliably transmitted even when the placement position of the first input button 210 and the actual actuation position of the main valve cartridge 310 are somewhat misaligned, thereby allowing the internal layout of the device as a whole to be configured more efficiently.

The second rotating lever 240, like the first rotating lever 230, is rotatably arranged within the internal accommodation space 130 of the main body portion 100 at a position adjacent to the main valve cartridge 310, and serves as a member that transmits the actuating force of the second input button 220 to the fluid control and delivery portion 300.

The second rotating lever 240 is also formed to rotate about the rotating support, and rotates in response to the pressing of the second input button 220 so as to selectively actuate another operational portion of the main valve cartridge 310. In other words, when the user presses the second input button 220, the second rotating lever 240 rotates, and the acting surface of the second rotating lever may push the corresponding control portion of the main valve cartridge 310 to open or regulate the corresponding flow path.

In particular, when the second rotating lever 240 is configured to be interlocked with the actuation system corresponding to the irrigation function, the second rotating lever is mechanically linked with the rotation transmission portion 420 of the dial-type continuous irrigation control portion 400, and may contribute to selectively maintaining the pressed state of the second input button 220. In other words, the second rotating lever 240 may not only transmit the button force but also function as a part of the internal actuation system related to maintaining a continuous irrigation state and regulating the irrigation flow rate.

The first rotating lever 230 and the second rotating lever 240 may be arranged independently or separated according to corresponding fluid functions, transmitting the force from each input button to different control portions of the main valve cartridge 310, thereby selectively actuating suction and irrigation functions. By employing this pair of rotating lever structures, the present disclosure provides a more stable operational feel, greater freedom in internal component layout, and, in particular, facilitates interlocking with the dial-type continuous irrigation control portion 400 on the irrigation side, compared to a simple structure that presses the valve directly with a button.

The fluid control and delivery portion 300 includes the main valve cartridge 310, the external fluid connection port 320, the front flow path delivery assembly 330, and an elastic support and retaining member 340.

The main valve cartridge 310 serves as the internal actuation unit at the center of the fluid control and delivery portion 300. The main valve cartridge 310 is disposed within the internal accommodation space 130 of the main body portion 100 and actually controls the suction and irrigation functions of the entire device. When the user presses the first input button 210 or the second input button 220 externally, the pressing force is ultimately transmitted through the first rotating lever 230 and the second rotating lever 240 to the component that actually opens or closes the fluid flow, which is the main valve cartridge 310.

Referring to FIGS. 9 and 10, the main valve cartridge 310 is formed as an elongated block body accommodated inside the main body, with the upper side linked to the operation input portion 200, the front side connected to the front flow path delivery assembly 330, and the rear or lower side connected to the external fluid connection ports 320.

Moreover, the main valve cartridge 310 does not merely serve as a passage for fluid but performs control functions to selectively actuate the suction and irrigation functions separately. That is, when one button is pressed, the suction system operates, and when another button is pressed, the irrigation system operates, with the internal fluid passages being selectively controlled.

Specifically, the main valve cartridge 310 may be configured to include both a suction control portion and an irrigation control portion within a single cartridge body. The suction control portion and the irrigation control portion may be selectively actuated in response to the operation of the first rotating lever 230 and the second rotating lever 240.

Accordingly, the main valve cartridge 310 may be regarded as a control portion that organizes the suction flow path and the irrigation flow path within the device to prevent them from crossing or interfering with each other, and converts the user’s input operations into actual fluid control actions. In particular, in the present disclosure, since the actuation system corresponding to the irrigation function may be linked with the control dial 410 and the rotation transmission portion 420, the main valve cartridge 310 may be operated not only for simple on/off control but also to maintain a continuous irrigation state and to respond to changes in its opening amount.

In other words, the main valve cartridge 310 controls both the suction system, which is in communication with an external suction source, and the irrigation system, which is in communication with an external irrigation fluid source. Depending on the user’s button and dial operations, the irrigation fluid may be delivered through the front flow path delivery assembly 330 toward the distal-end coupling portion 500 and the replaceable distal-end portion 600, while the suction system may be selectively operated to connect the replaceable distal-end portion 600 to the external suction source. Thus, the main valve cartridge 310 may be understood as a mediator in the surgical suction and irrigation device of the present disclosure, connecting button operations, dial adjustments, fluid opening/closing, and distal-end delivery.

The external fluid connection port 320 connects the main valve cartridge 310 to external fluid sources and is exposed externally through the rear fluid port support portion 170 of the main body portion 100. The external fluid connection port 320 is the connector that physically connects the suction line or irrigation fluid supply line coming from outside the device.

Referring to FIGS. 6, 9, and 10, the external fluid connection port 320 is formed as a protruding connection structure on the lower or rear side of the main body, and the user may couple an external hose or supply line to this port to provide the suction force and irrigation fluid supply required by the device. In the present disclosure, a plurality of external fluid connection ports 320 may be provided, with each port configured to correspond to a different fluid system, such as the suction system and the irrigation system.

The external fluid connection port 320 is not merely an accessory connector; rather, it serves as the starting point for fluid supply and discharge necessary for the device to operate in an actual surgical setting. Fluid supplied from the outside or suction pressure first enters the device through the external fluid connection port 320, is then selectively controlled by the main valve cartridge 310, and subsequently delivered to the distal-end portion along the front flow path delivery assembly 330. Therefore, the external fluid connection port 320 serves as the primary interface through which the device of the present disclosure connects to external systems, while also providing a structure that connects the internal fluid control mechanisms and distal-end operating components to the actual clinical environment.

The front flow path delivery assembly 330 is connected to the front of the main valve cartridge 310 and arranged to communicate fluidically with the distal-end coupling portion 500. The front flow path delivery assembly 330 may be understood as being configured such that, in the irrigation system, the irrigation fluid is delivered toward the replaceable distal-end portion 600, and in the suction system, a flow path is formed between the replaceable distal-end portion 600 and the external suction source, allowing blood, irrigation fluid, or other body fluids at the surgical site to be aspirated.

In the embodiments illustrated in FIGS. 8-10, the front flow path delivery assembly 330 may be formed in a shape including a front coupling head and a plurality of tubular members extending rearward. Particularly, in the illustrated embodiment, the assembly may be understood as being configured with a pair of spaced, curved tubular members, thereby providing an embodiment in which flow paths corresponding to the suction system and the irrigation system are formed.

The front flow path delivery assembly 330 is disposed at the front portion inside the main body and is connected to the rear of the distal-end coupling portion 500, having a tubular structure that bends downward and extends forward. In the illustrated embodiment, the assembly is illustrated as including multiple curved tubular members, which may be understood as a configuration that allows fluids corresponding to the suction function and the irrigation function to be delivered forward while remaining separated from each other.

In addition, the front flow path delivery assembly 330 not only performs the function of fluid delivery but may also structurally support the front positions of the distal-end coupling portion 500 and the replaceable distal-end portion 600. In other words, the front flow path delivery assembly 330 allows fluids delivered from the main valve cartridge 310 to move forward without loss, while simultaneously providing a support base to ensure that the distal-end coupling portion 500 maintains a stable position at the front of the main body.

Therefore, the front flow path delivery assembly 330 serves not only as a “pathway for fluid” within the device of the present disclosure but also as an “internal frame that supports the front distal-end structure”. In particular, since the present disclosure is based on the use of the replaceable distal-end portion 600, the front flow path delivery assembly 330 is significant in that, in conjunction with the distal-end coupling portion 500, it helps maintain a relatively consistent internal fluid path even after repeated replacement of the distal-end.

The elastic support and retaining member 340 is disposed beneath or adjacent to the front flow path delivery assembly 330 and is configured to elastically support the front flow path delivery assembly 330 or stabilize its connection state.

Referring to FIGS. 8-10, the elastic support and retaining member 340 is illustrated in the form of a coil spring and is disposed beneath the front flow path delivery assembly 330. That is, the elastic support and retaining member 340 serves as a spring member that prevents the front flow path delivery assembly 330 from being too loosely wobbly or, conversely, excessively rigid. By means of this member, the front flow path delivery assembly 330 may be supported with a certain degree of elasticity, resulting in a more stable connection with the main valve cartridge 310 and the forward distal-end structures.

In addition, the elastic support and retaining member 340 is not limited to a simple return spring; it may absorb assembly tolerances, ensure a tight connection, and help maintain the front flow path delivery assembly 330 in a relatively stable position even during repeated use. The elastic support and retaining member 340 may be understood as an elastic member in the form of a coil or torsion spring, which provides elastic restoring force or preload to the front flow path delivery assembly 330, thereby contributing to the maintenance of the reference position of the front flow path delivery assembly 330 and the stability of its connection with the main valve cartridge 310.

The dial-type continuous irrigation control portion 400 includes the control dial 410 and the rotation transmission member 420.

The control dial 410 is an adjustment member rotatably disposed on the side dial exposure and support portion 150 of the main body portion 100 so as to be manually operable from the outside. The control dial 410 may be rotated by the user in a state where the input button corresponding to the irrigation function is pressed, and the internal actuation system is selectively maintained according to the rotation, thereby establishing a continuous irrigation state.

More specifically, the control dial 410 is not limited to simply turning the irrigation function on or off; rather, it may vary the support position or the pressing force on the internal actuation system according to the amount of rotation, thereby adjusting the degree of opening of the irrigation flow path. As a result, the user may maintain irrigation without continuously pressing the input button corresponding to the irrigation function, and may finely adjust the flow rate by further rotating or releasing the control dial 410 as needed. In other words, the control dial 410 serves as an external operating interface that allows the user to directly rotate it from the side of the device to maintain continuous irrigation and control the flow rate.

The rotation transmission member 420 is an internal linkage component that transmits the rotational motion of the control dial 410 to the irrigation-side actuation system inside the device. The rotation transmission portion 420 is configured to transmit the rotational force of the control dial 410, when rotated, to at least one of the corresponding operating elements of the irrigation function, such as a rotating lever, button actuation system, or the main valve cartridge 310, thereby restricting the return of the actuation system or maintaining it in a pressurized state.

As a result, even when the user releases the input portion corresponding to the irrigation function, the internal actuation system does not immediately return to its original position, thereby allowing the irrigation flow path to remain open and enabling continuous irrigation. In addition, the rotation transmission portion 420 may vary the support position or pressing force of the internal actuation system according to changes in the rotation amount of the control dial 410, thereby changing the opening degree of the irrigation control portion of the main valve cartridge 310, and consequently, the irrigation flow rate delivered toward the replaceable distal-end portion 600 may be varied. In other words, the rotation transmission portion 420 can be understood as an internal transmission structure that conveys the movement of the control dial 410 externally rotated by the user to the internal irrigation actuation system, thereby implementing both the continuous irrigation maintenance function and the flow rate control function.

The distal-end coupling portion 500 includes a main-body-side female coupling portion 510, a rotary locking sleeve 520, and a distal-end insertion guide portion 530.

Referring to FIGS. 6-10, the main-body-side female coupling portion 510 serves as a coupling receiving portion at the center of the distal-end coupling portion 500 and is provided at the front side of the main body portion 100, where the proximal connector portion of the replaceable distal-end portion 600 is inserted.

The main-body-side female coupling portion 510 may be understood, as the name suggests, as an female coupling structure formed on the main body, and functions as the reference coupling portion that first engages when connecting the replaceable distal-end portion 600 to the main body. During use, the replaceable distal-end portion 600 is inserted axially toward the main-body-side female coupling portion 510, and in this process, the connector of the replaceable distal-end portion 600 is received within the main-body-side female coupling portion 510, thereby establishing a connected state in which the fluids can communicate with each other.

In addition, the main-body-side female coupling portion 510 is arranged to communicate with the front end of the front flow path delivery assembly 330, so that when the replaceable distal-end portion 600 is coupled, the corresponding flow paths are aligned and a fluid-communicating state is established. In this case, an annular sealing portion, such as an O-ring, may be interposed between the main-body-side female coupling portion 510 and the proximal connector portion of the replaceable distal-end portion 600 to suppress fluid leakage. Furthermore, the main-body-side female coupling portion 510 is not merely a hole for inserting the distal end; it functions as a reference coupling portion that defines the precise position of the replaceable distal-end portion 600, ensuring that after insertion, a stable fluid connection is maintained.

The rotary locking sleeve 520 is rotatably arranged around the outer side or periphery of the main-body-side female coupling portion 510 and serves as a locking member that selectively maintains or releases the coupling state of the replaceable distal-end portion 600.

In the embodiment illustrated in FIGS. 8 and 9, the rotary locking sleeve 520 is formed as an annular member surrounding the outer periphery of the main-body-side female coupling portion 510 and arranged to rotate roughly coaxially with the main-body-side female coupling portion 510. Moreover, by rotating the rotary locking sleeve 520, an internal engagement portion may be configured to engage or disengage with a corresponding engagement portion of the replaceable distal-end portion 600.

After inserting the replaceable distal-end portion 600 into the main-body-side female coupling portion 510, the user rotates the rotary locking sleeve 520 to prevent the distal-end portion from detaching. Conversely, rotating the sleeve in the opposite direction releases the distal end, allowing it to be detached. Accordingly, the rotary locking sleeve 520 may be configured to rotate between a release position and a locking position, and in the locking position, it restricts axial disengagement of the replaceable distal-end portion 600, preventing the distal end from inadvertently coming off during use. This configuration allows the user to quickly and easily replace the replaceable distal-end portion 600 while ensuring a stable usage state after coupling, with reduced risk of wobbling or disengagement.

In addition, the rotary locking sleeve 520 helps maintain a consistent coupling between the main-body-side female coupling portion 510 and the replaceable distal-end portion 600 even during repeated attachment and detachment, thereby enabling faster and more accurate replacement of the distal end during surgery. In other words, the rotary locking sleeve 520 is not merely an external cover, but functions as a locking mechanism that determines whether the replaceable distal-end portion 600 remains properly coupled.

The distal-end insertion guide portion 530 is a guide structure that facilitates the smooth insertion of the replaceable distal-end portion 600 into the main-body-side female coupling portion 510. In other words, when the user pushes the replaceable distal-end portion 600 toward the main body, the distal-end insertion guide portion 530 naturally aligns the insertion direction and guides the insertion of the proximal connector portion into the precise position within the main-body-side female coupling portion 510.

As illustrated in FIGS. 6 and 10, the distal-end insertion guide portion 530 may be formed as an enlarged or tapered portion provided around the front periphery of the main-body-side female coupling portion 510, thereby allowing the proximal connector portion of the replaceable distal-end portion 600 to be naturally guided toward the central axis during the initial stage of insertion.

Accordingly, the user does not need to forcibly align the distal end at an angle and may insert the distal end relatively naturally in the axial direction. Such a distal-end insertion guide portion 530 reduces alignment errors during the insertion process and more accurately aligns the coupling position between the proximal connector portion and the main-body-side female coupling portion 510, thereby contributing to improved stability of the fluid connection and reliability of sealing formation.

In particular, in order for the replaceable distal-end portion 600 to be coupled in the same manner through a common coupling interface, regardless of whether it is in the form of a flexible tube or a rigid tubular member, a guide structure that establishes the direction and position at the initial stage of insertion is important, and the distal-end insertion guide portion 530 may be understood as performing this function. As a result, the distal-end insertion guide portion 530 may function as an auxiliary guide that facilitates insertion of the replaceable distal-end portion 600, improves coupling accuracy with the main-body-side female coupling portion 510, and assists smooth locking by the rotary locking sleeve 520.

In addition, the robotic instrument coupling portion 10 (see FIGS. 1-5) may be provided on an outer side of a distal portion of the replaceable distal-end portion 600, and the robotic instrument coupling portion 10 may be selectively coupled to an end portion of the jaw or the grip member of the robotic surgical instrument or the laparoscopic instrument, such that the position of the distal portion of the replaceable distal-end portion 600 is controlled in accordance with manipulation of the instrument.

The replaceable distal-end portion 600 is a terminal working portion that is selectively coupled to the front of the main body portion 100 and extends to the actual surgical site.

The replaceable distal-end portion 600 may include a proximal connector portion configured for coupling with the distal-end coupling portion 500, and the proximal connector portion may be inserted into the main-body-side female coupling portion 510 to establish both a mechanical connection and fluid communication. The replaceable distal-end portion 600 may include the proximal connector portion inserted into the main-body-side female coupling portion 510 and a slender tube or conduit extending forward from the connector. In the illustrated embodiment, a rigid tubular distal-end portion is exemplified, and a distal end thereof may be formed in a bent shape to facilitate access to a surgical site.

In the present disclosure, the replaceable distal-end portion 600 is detachably connected to the distal-end coupling portion 500, thereby allowing different distal-end configurations suitable for various surgical conditions to be selectively applied while using the same main body. In other words, the replaceable distal-end portion 600 is not merely a component that extends the overall length of the device, but is configured to allow suction or irrigation to reach the surgical site and to be replaceable with a configuration suitable for the position of the surgical site, the access path, and the confined working space.

In addition, the replaceable distal-end portion 600 may be inserted into and coupled with the main-body-side female coupling portion 510 of the distal-end coupling portion 500, and may be configured such that the coupled state is maintained by the rotary locking sleeve 520. In other words, the replaceable distal-end portion 600 is not merely inserted into the front of the main body, but may have a structure in which it is inserted in an axial direction and then securely locked by rotation of the rotary locking sleeve 520.

In this case, the distal-end insertion guide portion 530 may serve to guide the replaceable distal-end portion 600 so that it is inserted into the main-body-side female coupling portion 510 with relatively high accuracy. Accordingly, when replacing the distal end, the user may remove the existing distal end in an axial direction, insert another distal end, and operate the rotary locking sleeve 520 to maintain the coupled state. With such a configuration, the distal end may be replaced relatively quickly, and a stable coupling state may be secured such that the distal-end portion does not wobble or easily disengage even after replacement.

The replaceable distal-end portion 600 has a length through which internal fluid can pass and forms an actual working end at a surgical site. In other words, in the irrigation system, irrigation fluid controlled by the fluid control and delivery portion 300 is delivered toward the replaceable distal-end portion 600 via the front flow path delivery assembly 330 and may be discharged at the distal end, while in the suction system, the replaceable distal-end portion 600 is in fluid communication with an external suction source, allowing blood, irrigation fluid, or other body fluids at the surgical site to be aspirated through the replaceable distal-end portion 600.

Therefore, the replaceable distal-end portion 600 is not merely an external rod, but may be regarded as a distal end portion in which actual functions are performed at the surgical site while forming a final delivery path of fluid. In particular, in the present disclosure, since the replaceable distal-end portion 600 is configured to be detachable from the main body, it is significant in that the rigidity or flexibility, bending characteristics, and directionality of the distal end may be selectively adapted to the surgical conditions while using the same main body.

Meanwhile, the replaceable distal-end portion 600 is not limited to a single configuration, but may include a plurality of distal-end portion types having a common coupling interface. For example, in another embodiment, a flexible tube-type distal-end portion may be applied, and in yet another embodiment, a rigid tubular distal-end portion may be applied. Although a rigid tubular distal-end portion is exemplarily illustrated in the drawings, the technical concept of the present disclosure is not limited thereto.

The replaceable distal-end portion 600 may be commonly coupled to the same distal-end coupling portion 500 while being selectively provided in different configurations depending on the position of the surgical site, the access direction, and the required flexibility or directionality. In particular, a flexible tube-type distal-end portion may be used as a primary embodiment of the present disclosure, and may be advantageously employed in robotic surgery where flexible access along a curved path is required using the articulated motion of a robotic instrument. Meanwhile, a rigid tubular distal-end portion may be selectively used in cases where higher directionality or support is required, such as in laparoscopic surgery, or where a rigid shaft is needed.

Hereinafter, an operating sequence of the present disclosure according to the above configuration will be described with reference to FIGS. 1-10.

First, the user selects the replaceable distal-end portion 600 suitable for the surgical purpose and couples the replaceable distal-end portion 600 to the front of the main body portion 100. In this case, the replaceable distal-end portion 600 is inserted in an axial direction toward the main-body-side female coupling portion 510 of the distal-end coupling portion 500, and while being aligned by the distal-end insertion guide portion 530, the rotary locking sleeve 520 is rotated to maintain the coupled state.

In particular, when a flexible tube-type distal-end portion is used in robotic surgery, a jaw of a robotic surgical instrument may be coupled to the robotic instrument coupling portion 10 provided on an outer side of the distal portion of the replaceable distal-end portion 600. In this case, since the distal portion of the flexible tube-type distal-end portion moves together in accordance with position control of the jaw, the surgeon may more efficiently perform access to the surgical site, as well as suction and irrigation, without separately holding a flexible suction instrument.

At the same time, the external suction source and the irrigation fluid supply source are connected to the external fluid connection port 320 at the rear, thereby establishing a ready state in which the main valve cartridge 310 and the front flow path delivery assembly 330 are in fluid communication. In this state, since the front flow path delivery assembly 330 is elastically supported by the elastic support and retaining member 340, the internal connection state within the main body and the structure at the front distal end may be maintained more stably.

Next, in a default standby state, the first input button 210 and the second input button 220 are not pressed, and the control dial 410 is also positioned in a release state. At this time, the main valve cartridge 310 maintains flow paths corresponding to suction and irrigation in a closed or restricted state by default, such that no continuous fluid flow is generated through the replaceable distal-end portion 600. That is, even though the device is connected to an external fluid source, it remains in the standby state until the user operates the operation input portion 200.

When the user intends to perform a suction function, the user presses the input button corresponding to the suction function. For example, when the first input button 210 is configured to correspond to the suction function, a pressing force of the first input button 210 is transmitted to the main valve cartridge 310 through the first rotating lever 230, and accordingly, the suction control portion of the main valve cartridge 310 is actuated to open the suction flow path.

Accordingly, a suction force from the external fluid connection port 320 connected to the external suction source is transmitted through the main valve cartridge 310, the front flow path delivery assembly 330, and the distal-end coupling portion 500 to the replaceable distal-end portion 600, such that blood, body fluid, or irrigation fluid may be aspirated at a distal end of the distal-end portion. When the user releases the first input button 210, the operation of the first rotating lever 230 is released, and the suction control portion of the main valve cartridge 310 returns to its original position, thereby stopping the suction operation.

Meanwhile, when the user intends to perform an irrigation function, the user presses the input button corresponding to the irrigation function. For example, when the second input button 220 is configured to correspond to the irrigation function, a pressing force of the second input button 220 is transmitted to the irrigation control portion of the main valve cartridge 310 through the second rotating lever 240, and accordingly, the irrigation flow path may be opened.

Accordingly, the irrigation fluid supplied from the external fluid connection port 320 connected to the external irrigation fluid source is delivered through the main valve cartridge 310, the front flow path delivery assembly 330, and the distal-end coupling portion 500 to the replaceable distal-end portion 600, and saline or irrigation fluid may be discharged at the distal end. In this case as well, when the user releases the second input button 220, the irrigation control portion of the main valve cartridge 310 returns to its original position, and the irrigation operation may be accordingly stopped.

The distinctive operation of the present disclosure becomes even clearer in the continuous irrigation maintenance and flow rate adjustment functions. When the second input button 220 corresponding to the irrigation function is pressed and the user rotates the control dial 410, the rotational force is transmitted via the rotation transmission portion 420 to the internal actuation system. As a result, the portion corresponding to the second rotary lever 240 or the irrigation control portion of the main valve cartridge 310 is mechanically supported or its return is restricted, allowing the irrigation flow path to remain open even when the user releases the second input button 220. In other words, the second input button 220 functions as the input to initiate continuous irrigation, while the control dial 410 and the rotation transmission portion 420 serve as maintaining means to sustain this state.

Furthermore, the control dial 410 does not merely maintain the irrigation state; it can also adjust the flow rate according to the amount of rotation. The more the control dial 410 is rotated, the internal support position or the pressing force via the rotation transmission portion 420 changes, thereby altering the opening degree of the irrigation flow path in the main valve cartridge 310. Consequently, the amount of irrigation fluid discharged from the distal end of the replaceable distal-end portion 600 may increase or decrease. Conversely, when the user rotates the control dial 410 in the release direction, the mechanical maintenance by the rotation transmission portion 420 is released, the actuation system corresponding to the irrigation function returns to its original position, and the irrigation operation is terminated.

Finally, when it is desired to change the distal-end configuration during surgery or to detach the distal end after use, the rotary locking sleeve 520 is rotated in a release direction to release the locked state of the replaceable distal-end portion 600, and then the replaceable distal-end portion 600 may be axially separated from the main-body-side female coupling portion 510.

Thereafter, when another replaceable distal-end portion 600 having a different configuration is reinserted along the distal-end insertion guide portion 530 and the rotary locking sleeve 520 is rotated in the locking direction, the device may be reused with a new distal end while maintaining the same main body portion 100. As described above, the surgical suction and irrigation device according to the present disclosure is configured such that selective suction and irrigation operation by button input, continuous irrigation maintenance and flow rate control by a dial, and rapid replacement of the distal end are organically achieved within a single device.

The scope of protection of the present disclosure is not limited to the descriptions and expressions of the embodiments explicitly described above. Furthermore, it is noted that the scope of protection of the present disclosure should not be limited by modifications or substitutions that would be apparent to those skilled in the art to which the present disclosure pertains.

What is claimed is:

1. A surgical suction and irrigation device, comprising:
a main body;
an extension portion fluidically connected to the main body and at least partially elastically bendable so as to extend to a surgical site; and
an instrument coupling portion provided on an outer side of a distal portion of the extension portion and configured to be selectively coupled with a jaw or grip member of a robotic surgical instrument or a laparoscopic instrument,
wherein the main body is configured to perform suction or irrigation through the extension portion, and
a position of the distal portion of the extension portion is controlled according to position control of the jaw or the grip member when the jaw or the grip member is coupled to the instrument coupling portion.

2. The surgical suction and irrigation device of claim 1, wherein the main body includes a first input button and a second input button corresponding to a suction function and an irrigation function, respectively.

3. The surgical suction and irrigation device of claim 2, wherein the main body further includes a first rotating lever and a second rotating lever configured to transmit actuating forces of the first input button and the second input button to a fluid control portion of the main body, and
the first and second rotating levers each include a rotating support and an inclined acting surface to convert a button pressing displacement into a fluid control actuation displacement.

4. The surgical suction and irrigation device of claim 1, wherein the instrument coupling portion includes a distal connecting member provided at the distal portion of the extension portion, and a fitting-type coupling portion formed in the distal connecting member configured to allow insertion and removal of an end portion of the jaw or grip member.

5. The surgical suction and irrigation device of claim 4, wherein the fitting-type coupling portion is formed in a pocket shape.

6. The surgical suction and irrigation device of claim 4, wherein the fitting-type coupling portion includes a toothed portion on an inner side, outer side, or both sides to increase a frictional force with the jaw or grip member.

7. The surgical suction and irrigation device of claim 6, wherein the toothed portion has a triangular or right-triangular cross-section to prevent detachment after insertion.

8. The surgical suction and irrigation device of claim 1, wherein the instrument coupling portion further includes a through-type coupling portion configured to allow an end portion of the jaw or grip member to pass through and be separated.

9. The surgical suction and irrigation device of claim 8, wherein the through-type coupling portion includes a latch projection line to prevent the jaw or grip member from easily disengaging after passing through.

10. The surgical suction and irrigation device of claim 1, wherein a distal end of the extension portion includes at least one through hole.

11. The surgical suction and irrigation device of claim 1, wherein the extension portion is configured to extend to the surgical site through a port or hole separate from a primary port through which a robotic arm passes during robotic surgery.

12. The surgical suction and irrigation device of claim 1, wherein the extension portion is formed as a replaceable distal-end portion detachably coupled to the main body.

13. The surgical suction and irrigation device of claim 12, wherein the main body includes a distal-end coupling portion to which the replaceable distal-end portion is coupled,
the distal-end coupling portion includes a main-body-side female coupling portion, a rotary locking sleeve, and a distal-end insertion guide portion, and
the replaceable distal-end portion is inserted axially into the main-body-side female coupling portion, and then, is configured such that a coupled state is maintained by rotation of the rotary locking sleeve.

14. The surgical suction and irrigation device of claim 13, wherein the replaceable distal-end portion includes a proximal connector portion inserted into the main-body-side female coupling portion, and
a sealing portion is interposed between the proximal connector portion and the main-body-side female coupling portion for fluid sealing.

15. The surgical suction and irrigation device of claim 12, wherein the replaceable distal-end portion includes a plurality of distal-end portions having a common coupling interface, and
the plurality of distal-end portions includes a flexible tube-type distal-end portion with the instrument coupling portion and a rigid tubular distal-end portion.

16. The surgical suction and irrigation device of claim 1, wherein the main body further includes a dial-type continuous irrigation control portion configured to mechanically maintain an actuation input state corresponding to an irrigation function and vary an irrigation flow rate according to a rotation amount of a dial.

17. The surgical suction and irrigation device of claim 16, wherein the dial-type continuous irrigation control portion includes a control dial rotatably arranged on a side surface of the main body and a rotation transmission portion configured to transmit rotation of the control dial to an internal actuation system.

18. The surgical suction and irrigation device of claim 17, wherein the rotation transmission portion is configured to limit return of the actuation input state corresponding to the irrigation function so that a continuous irrigation state is maintained even when a user releases an input portion corresponding to the irrigation function, and
a support position or pressing force of the actuation system corresponding to the irrigation function is changed according to a rotation amount of the control dial so that an opening amount of an irrigation flow path is variable.

19. The surgical suction and irrigation device of claim 1, wherein the main body further includes a fluid control and delivery portion including a main valve cartridge, a plurality of external fluid connection ports, and a front flow path delivery assembly extending from the main valve cartridge toward the extension portion.

20. The surgical suction and irrigation device of claim 19, wherein the main valve cartridge includes a suction control portion and an irrigation control portion in a single cartridge body, and
the front flow path delivery assembly is configured to form a suction flow path and an irrigation flow path that are separated from each other, and to be elastically supported or to have a connection state thereof stabilized by an elastic support and retaining member.
